(19) United States
(12) Patent Application Publication
(10) Pub. No.: US 2023/0248472 A1
(43) Pub. Date: Aug. 10, 2023

(54) SYSTEM AND METHOD FOR TREATMENT OF TOOTH DECAY, INFECTION OR ABSCESS AND APPARATUS RELATED THERETO

(71) Applicant: Andrew Cosentino, TUCSON, AZ (US)

(72) Inventor: Andrew Cosentino, TUCSON, AZ (US)

(21) Appl. No.: 18/137,615

(22) Filed: Apr. 21, 2023

Related U.S. Application Data
(63) Continuation of application No. 17/567,781, filed on Jan. 3, 2022, now abandoned.
Application No. 17/567,781 is a continuation of application No. 16/201,817, filed on Nov. 27, 2018, now abandoned.
(60) Provisional application No. 62/647,452, filed on Mar. 23, 2018.
(60) Provisional application No. 62/729,343, filed on Sep. 10, 2018.

(51) Int. Cl. A61C 5/00

(52) U.S. Cl. CPC A61C 5/007

(57) ABSTRACT

Disclosed is a bite blocker device for preventing occlusion of an affected dentition. The channel of the bite block follows and engages a portion of the user's upper teeth, while a base portion, opposite the channel portion, follows and engages a portion of the user's lower teeth. The base portion and the channel are separated forming a gap between the user's upper teeth and lower teeth. The bite block is placed substantially opposite the affected tooth or teeth, the gap formed by the bite block preventing occlusion of the affected teeth.

### CROSS REFERENCE TO RELATED APPLICATIONS

This application is a continuation of U.S. patent application Ser. No. 17/567,781 filed on Jan. 3, 2022, which in turn is a continuation of U.S. patent application Ser. No. 16/201,817 filed on Nov. 27, 2018, which application claims the benefit of U.S. Provisional Patent Application Serial Nos. 62/647,452 filed Mar. 23, 2018 and 62/729,343 filed Sep. 10, 2018. All of the foregoing applications are hereby incorporated by reference in their entirety as if fully set forth herein.

### COPYRIGHT NOTICE

This disclosure is protected under United States and/or International Copyright Laws. © 2018 Andrew Cosentino. All Rights Reserved. A portion of the disclosure of this patent document contains material which is subject to copyright protection. The copyright owner has no objection to the facsimile reproduction by anyone of the patent document or the patent disclosure, as it appears in the Patent and/or Trademark Office patent file or records, but otherwise reserves all copyrights whatsoever.

### FIELD OF THE DISCLOSURE

This disclosure relates generally to the field of dental devices, and more specifically to a customizable bite block device.

### BACKGROUND

Every year in the USA, there are an estimated 15 million persons who will require a root canal procedure by an endodontist, or a dentist. And, every year in the USA, there are an estimated 22 million persons who will require an extraction by a dental provider. These root canals and extractions are most often related to an abscessed tooth, or non-retractable pain of a different origin.

The pain associated with an abscessed tooth is extreme, sharp, debilitating, and most often brought on by direct pressure on the affected tooth. Therefore occlusion of the dentition, whereby contact is made between the affected tooth, and non-affected tooth, or teeth on the opposing element of the jaw is met with sharp, extreme pain. For example: If the patient has an upper 2nd molar on the left side of the mouth that has an abscessed root, when it comes in contact with the lower 2nd molar on the left side of the mouth, or it's adjoining teeth, the slightest contact pressure creates the pain response. Patients often refer to it as a “10” on the pain scale, with “10” signifying the most extreme pain imaginable, and “0” being no pain at all.

In the awake state, most persons can avoid the pain by avoiding occlusion of the dentition. They will carry themselves about with a slack jaw, whereby the lower jaw (mandible) is held in the slack position (mouth slightly open), thereby inhibiting, or preventing occlusion of the upper and lower teeth.

In the sleeping state, the unconscious, or semi-conscious mind is unable to direct the jaw to remain in the slack, or open position. Inadvertent contact throughout the night causes repeat, frequent onset of this extreme pain as the affected tooth is brought in contact with the opposing tooth or teeth. The patient is therefore deprived of needed rest, and is brought to a state of exhaustion as they suffer from these repeat onslaughts of pain.

This inability to avoid the “closed” position of the dentition (whereby the upper and lower teeth make contact), most notably experienced while the patient is attempting to sleep, is compounded by the fact that many patients cannot gain immediate access to an endodontist or a dentist who will perform the root canal or extraction that brings the ultimate resolution to the pain.

Patients awaiting an appointment that is often days away are forced to consume narcotics, and or opiates such as Tylenol with Codeine or Vicodin. Even then, the pain associated with occlusion of the teeth often surpasses the ability of these drugs to suppress such pain. In addition, consumptions of these drugs are fraught with numerous potential adverse side effects such as overdose, suppression of the respiratory drive, constipation, and addiction, to name just a few.

Not only is the patient subjected to deprivation of sleep while awaiting an appointment, but once seen many dental providers may insist that the patient undertake a 7, or even 10 day course of antibiotics in an attempt to resolve the infection within the abscessed tooth before taking further action. During this time, the patient continues to suffer sleep deprivation as the pain associated with occlusion of the upper and lower teeth continues to plague the patient while in the unconscious or semi-conscious state.

When querying dental professionals, the same solution to night time rest is offered, which is the consumption of narcotics.

### SUMMARY OF THE INVENTION

The present disclosure addressed one or more problems identified in the background section with a Customizable Thermoplastic Hemi Bite Block (CTHBB) or bite blocker device.

The CTHBB can provide an emergency, short term strategy that prevents occlusion of the affected dentition until which time an endodontist or dentist can perform a root canal, or an extraction, or until which time the patient completes a course of antibiotic therapy in an attempt to resolve any infection. The bite block can be made of a thermoplastic compound allowing the device to be fitting by individuals, or a dental technician. This provides an at-home remedy to those experiencing dental pain, as well as an additional option for medical professionals when presented with a patient experiencing tooth pain or discomfort.

The bite block may be placed in either the right side of the mouth, or a mirror image can be placed in the left side of the mouth. The device is designed to be worn on the side opposite the point of pain, an abscessed tooth for example. The user, or anyone else, may heat the device, activating the thermoplastic, and then install the device before it cools completely in order to further customize the fit. The CTHBB is designed to be worn during the day, or during the evening to prevent occlusion of the upper and lower teeth on the opposite side of the mouth (where the affected tooth resides).

In various examples, the present invention comprises a channel, the channel configured to follow and engage a portion of the user's upper teeth. Opposite the channel is a base portion, the base portion configured to follow and engage a portion of the user's lower teeth, and wherein the base portion is separated from the channel portion by at least 5 mm forming a gap between the portion of the upper teeth and the portion of the lower teeth. Two walls extend upward from the sides of the channel. First, an anterior wall, wherein the anterior wall extends upward from the channel portion and is configured to be positioned on the outside of a portion of the patient's upper teeth and gums. Second, a posterior wall, wherein the posterior wall extends upward from the channel portion and is configured to be positioned on the inside of the portion of the patient's upper teeth and gums. Together the walls and channel substantially surround the user's upper teeth in order to hold the bite block in place. In various embodiments a portion of the walls and channel may be thermoplastic and activated to be further formed to the user's mouth.

### BRIEF DESCRIPTION OF THE DRAWINGS

In the drawings, identical reference numbers identify similar elements or acts. The sizes and relative positions of elements in the drawings may not be necessarily drawn to scale. For example, the shapes of various elements and angles may not be drawn to scale, and some of these elements may be arbitrarily enlarged or positioned to improve drawing legibility.

FIG. 1A depicts a front perspective view of an embodiment of the present invention;

FIG. 1B depicts a front perspective view of an alternative embodiment of the present invention short tongue wall embodiment;

FIG. 2A depicts a rear perspective view of an embodiment of the present invention;

FIG. 2B depicts a rear perspective view of an alternative embodiment of the present invention;

FIG. 3 depicts a top orthographic view of an embodiment of the present invention;

FIG. 4 depicts a bottom orthographic view of an embodiment of the present invention;

FIG. 5 depicts a back orthographic view of an embodiment of the present invention;

FIG. 6 depicts a left-side of an embodiment of the present invention;

FIG. 7 depicts a right-side of an embodiment of the present invention;

FIG. 8 depicts the right-half embodiment of the present invention in bite-down position in a partial frontal open-mouth view;

FIG. 9 depicts the right-half embodiment of the present invention in bite-down position of an open-mouth delineation in a perspective partial side view emphasizing the gap distance between upper and lower teeth on the oral cavity side not having the bite block; and

FIG. 10 depicts a mostly closed-mouth partial frontal view.

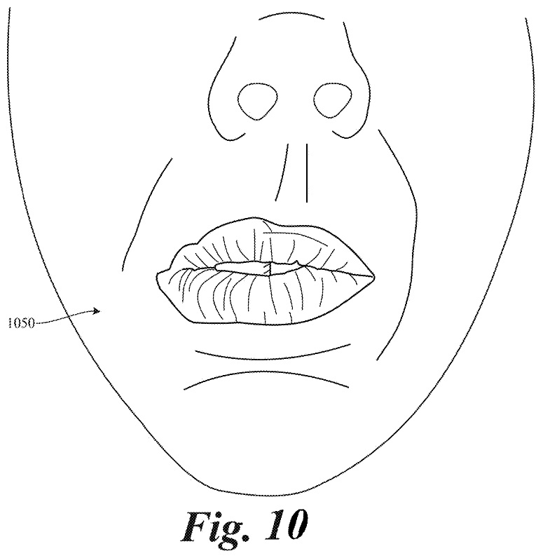

### DETAILED DESCRIPTION

In some aspects, the CTHBB or bite blocker is made of a thermoplastic compound. The device can be fitted to an individual by heating the device in hot water, thereby creating a malleable state. The device may then be fitted to the non-affected site (the side of the mouth that is not affected by the abscessed tooth). As the compound cools, the form becomes permanent. This process takes a matter of minutes to accomplish, and can be performed by either the individual or a dental technician.

The CTHBB may be placed in either the right side of the mouth, or a mirror image CTHBB can be placed in the left side of the mouth. If the device is placed in the right side of the mouth, the upper and lower teeth on the left side of the mouth are blocked from occlusion (or contact), thereby preventing the onset of pain associated with occlusion and the resultant contact pressure on an affected tooth. Hence, a right CTHBB prevents occlusion of the left dentition, and a left CTHBB prevents occlusion of the right dentition.

The CTHBB, by virtue of having been customized to the dentition of the patient, is seated firmly in place and is not subject to movement in the mouth. In the event the CTHBB, following custom thermoplastic fitting, is not firmly seated over the dentition, a denture adhesive cream, for example, may be applied to the inner channel of the CTHBB prior to seating the device over the teeth. This will provide added assurance that the device will not be subject to movement in the mouth. Various other or alternative adhesives may also be used in order to provide additional security. The adhesive may be applied for example, after the CTHBB has been formed to the user's mouth, or without custom forming.

In additional embodiments, the CTHBB may be formed based on a 3D scan of a user's mouth. The user's mouth can be scanned, resulting in a 3-dimensional model of the area to be protected by the CTHBB. Using the 3D model, a custom CTHBB may be constructed using various molding, CNC, pressure casting, 3D printing or other techniques. In such an embodiment, non-thermoplastic materials can be used, since custom forming is accomplished in the design and manufacturing process itself.

In some instances, the CTHBB can provide an emergency, short term strategy that prevents occlusion of the affected dentition until which time an endodontist or dentist can perform a root canal, or an extraction, or until which time the patient completes a course of antibiotic therapy in an attempt to resolve any infection.

The CTHBB can be worn during the day, or during the evening to prevent occlusion of the upper and lower teeth on the contra-lateral side of the mouth (where the affected tooth resides). The CTHBB may provide the patient a method of preventing occlusion of the affected teeth during the sleeping hours thereby providing the patient the opportunity to gain restful restorative sleep.

By virtue of the device preventing occlusion of the affected tooth, or teeth, thereby avoiding the painful stimuli of pressure on the affected tooth, the patient may be able to consume lower doses of narcotics, or may be able to avoid the consumption of narcotics all together (thereby lowering, or eliminating the potential adverse effects of consuming narcotics or opiates).

In some cases, the CTHBB or bite block device (e.g., the layer formed between the teeth), may be approximately 3-10 mm thick at its far anterior position, and 3-12 mm thick at it's far posterior position. When installed into the mouth of a patient, there may be approximately 2-10 mm of clearance between the upper and lower teeth. The device may be designed to target a clearance between the upper and lower dentition of between 3 to 9 mm, with 5-7 mm to be optimally comfortable, and to assure that the molars in the rear are not making contact. Depending on the size of a patient's teeth, mouth, jaw, etc., the above dimensions may vary anywhere between 5, 10, 20, or even 50 percent.

In additional embodiments, the posterior portion of the CTHBB may be raised by a few mm to approximate the height of the anterior portion.

In some aspects, the amount of material used to produce the pre-formed mold (e.g., thickness) may be, at least in part, selected to yield a different height of sidewall formed when the patient bites down on the pre-formed mold. This may be particularly useful for patients having smaller sized teeth, mouth, or jaw.

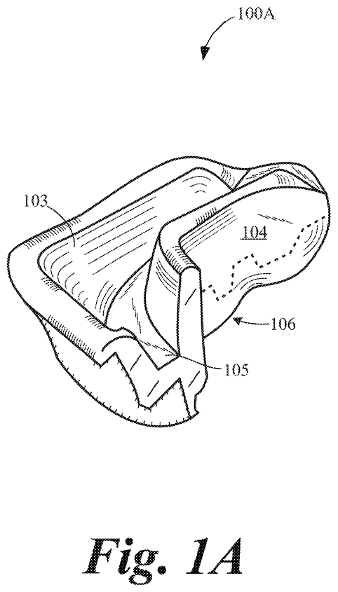

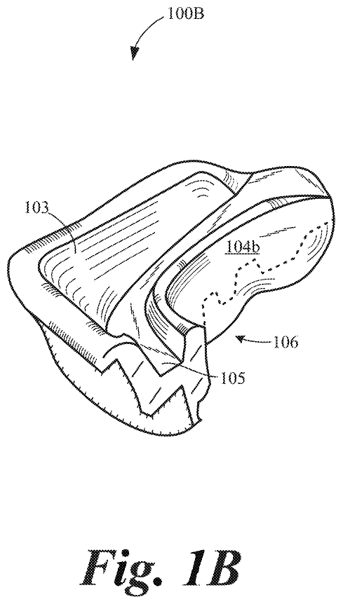

FIG. 1A depicts a front perspective view of an embodiment 100A of the present invention. As depicted the bite blocker includes an anterior wall 103 and a posterior wall 104. These walls are separated and form channel 105. Together, the space formed by the interior portions of the anterior wall 103, the posterior wall 104 and the channel 105 accepts the user's teeth and gums when installed. For example, the upper ridge of the anterior wall, when the bite block is installed, is located toward the upper portion of the user's outer facing gums, near the base of the upper lip. The overall height of the walls 103 and 104 may depend on the model or size offered. For example, an adult version may have walls of approximately 12-18 mm, whereas a model for younger or smaller users may have a range from 10-16 mm. In addition, the height of walls 103 and 104 relative to one another may change depending on the embodiment, for example, 100A depicts the walls of equal or nearly equal height. A taller posterior wall 104, for example, may help improve stability of the device. This is helpful where the user intends to wear the device while sleeping, the taller posterior wall 104 preventing the blocker from shifting while the user is unconscious.

The overall shape and contour of the channel 105 as well as the shape and contour of the walls 103 and 104 where they meet channel 105 can be augmented by the user during the thermo-forming process. The same is true of the underside of the bite block, 106. In some embodiments, including the one depicted in FIG. 1A, the underside 106 may include pre-molded portion to better form to the user's teeth. Such as, in the depicted example, the underside 106 is molded to better form to the user's lower teeth. These ridges in lower portion 106 can help increase the contact area of the device, improving comfort and stability. The contour and shape of the lower portion 106 can also be altered through the thermo-forming process, including where ridges are pre-molded.

FIG. 1B depicts an alternative embodiment 100B. This embodiment is very similar to 100A, except that here, the posterior wall 104 b is much shorter compared to anterior wall 103. The height of the posterior wall 104 b may be anywhere between that depicted in embodiments 100B and 100A, the depicted embodiments should not be seen as limiting. The shorter posterior wall 104 b may be more comfortable for some users, especially those who do not intend on wearing the blocker at night, or, alternatively, for those who intend to use an alternative means of increasing the bite blocker's security, for example, denture adhesive cream applied in channel 105.

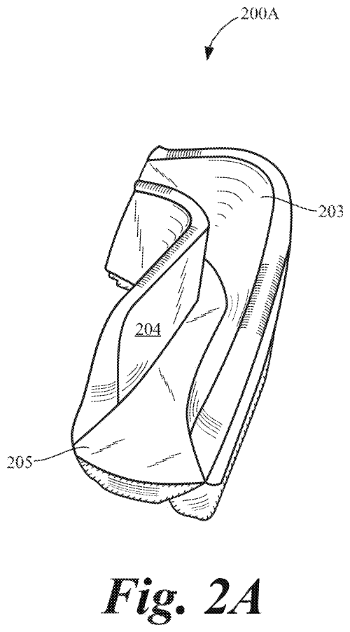

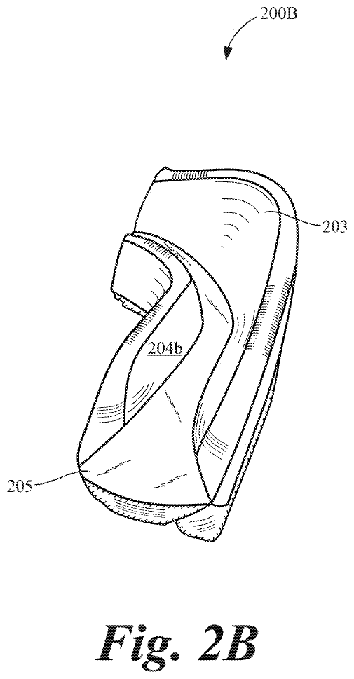

FIGS. 2A and 2B depicts a rear perspective view of embodiments 100A and 100B, here labeled as 200A and 200B. As depicted, the channel 205 may be shaped to progressively widen as the channel extends toward the back of the mouth in order to accommodate the wider tooth profiles. The walls, 203 and 204 may also progressively taper, eventually meeting the channel 205.

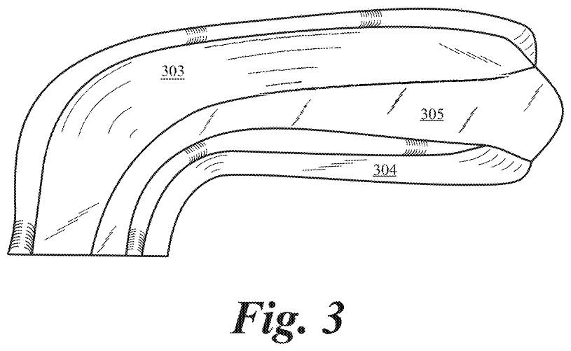

FIG. 3 provides a top down perspective of an embodiment of the present invention. As depicted, the upper portion of either wall 303 or 304 may be rounded, and may increase or decrease in thickness at various points. For example, additional thickness in the corner of the mouth may provide additional security and comfort. This perspective, again, shows where the profile of the channel 305 changes along its length to accommodate the change in tooth shape and profile.

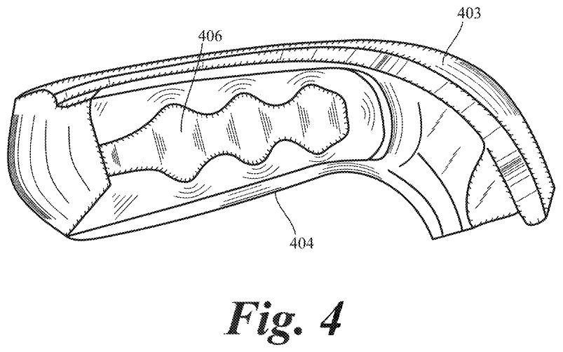

FIG. 4 provides a bottom up perspective of an embodiment of the present invention. As depicted, the bottom 406, may include ridges to accommodate the teeth opposite the channel. Bottom 406 may be constructed of different polymers in order to better accept the teeth. For example, the inner portion, as depicted, may be constructed of a softer more thermos-responsive material, while the perimeter portion may be more firm, providing stability. In various embodiments, the anterior wall, 403, may extend beyond the bottom 406. This can provide additional support and comfort, resting against the inner portion of the user's lip.

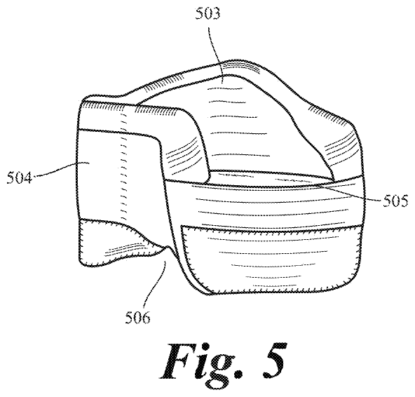

FIG. 5 depicts a rearward view of a bite block device. From this viewpoint, the thickness of the point from the channel 505 to the bottom 506 is demonstrable. In the depicted embodiment bite block device (e.g., the layer formed between the teeth) may be approximately 7.6 mm thick at its far anterior position, and 8.5 mm thick at it's far posterior position. When installed into the mouth of a patient, there may be approximately 6 mm of clearance between the upper and lower teeth. The device may be designed to target a clearance between the upper and lower dentition of between 5 to 7 mm to be optimally comfortable, and to assure that the molars in the rear are not making contact. It should be appreciated that the above dimensions are only given by way of example. Depending on the size of a patient's teeth, mouth, jaw, etc., the above dimensions may vary anywhere between 5, 10, 20, or even 50 percent. In some cases, different size bite blocker molds (pre-formed) may be designed for different patients to accommodate different teeth and mouth or jaw sizes, based on age, or other metric.

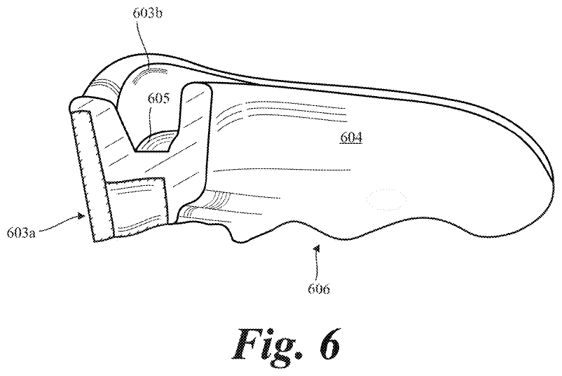

FIG. 6 depicts a side view of a right sided embodiment of the present invention. In the depicted embodiment, the anterior wall 603 is constructed of at least two different polymers, 603 b and 603 a. In such an embodiment, the outer portion, 603 a, may be constructed from a stiffer material to provide increased rigidity to the device in order to ensure it maintains proper shape and alignment when in use. Meanwhile, the inner-portion 603 b may be constructed of a softer, more pliable material in order to allow it to better conform to the user's teeth and gums. More specifically, in various embodiments, bite block device may be made from a variety of materials, including one or more thermoplastic compounds, such as found in commercially available sports mouth guards. In multilayer embodiments, such as the one depicted, the outer layer (e.g., corresponding to the part of the bite blocker that is positioned on the outside of the patient's teeth) may consist of a solid medical grade, FDA approved Polypropylene that adds structural support and strength. The inner layer (e.g., corresponding to the part of the bite blocker that is positioned in between/on the inside of the patient's teeth) may consist of any thermoplastic material approved by the Food and Drug Administration (FDA), that renders the bite blocker it's characteristic “boil and bite” feature. The most common thermoplastic material for this type of application is ethylene-vinyl acetate (EVA), but other materials may work as well and will be known to those in the art. The “boil and bite” characteristic is derived from the thermoplastic inner lining that softens when applied to hot water, then adapted to the individual's mouth by biting down on it and holding for typically 30 to 45 seconds. The use of a thermoplastic compound provides a self-adapting quality allowing the upper teeth to be seated more comfortably into the device by generating a custom mold. It also secures the teeth into the device much more adequately than the alternative of using a rigid, non-thermoplastic compound in the channel of the bite blocker.

While not depicted in this particular embodiment, the posterior wall 604 may also be constructed of one or more materials much in the same manner and for the same reasons as the anterior wall 603.

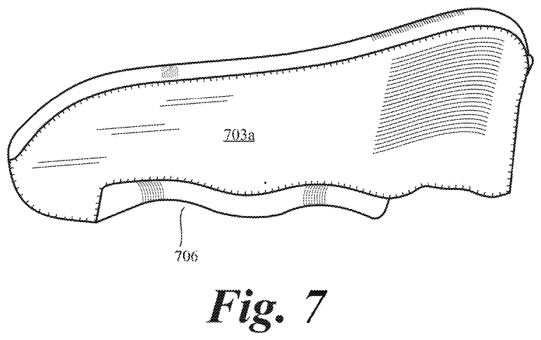

FIG. 7 depicts the side view of an embodiment of the present invention employing at least a second material for the outer portion of the anterior wall 703. As depicted, the preferably stiffer outer material 703 a may extend significantly rearward and may cover some or the entire anterior wall. As depicted, in some embodiments the stiffer portion 703 a may end prior to the upper edge. Where this is the case, the second material, preferably a softer material is used at the points of contact with the gums and teeth. The outer portion may be stylized, or formed with additional designs or arrangements to increase stiffness further or to be more pleasing to the eye. A third or more material may also be used along a portion of the outer wall 703 in order to provide additional stiffness. For example, a third material may be placed in a bracing fashion longitudinally along the anterior wall.

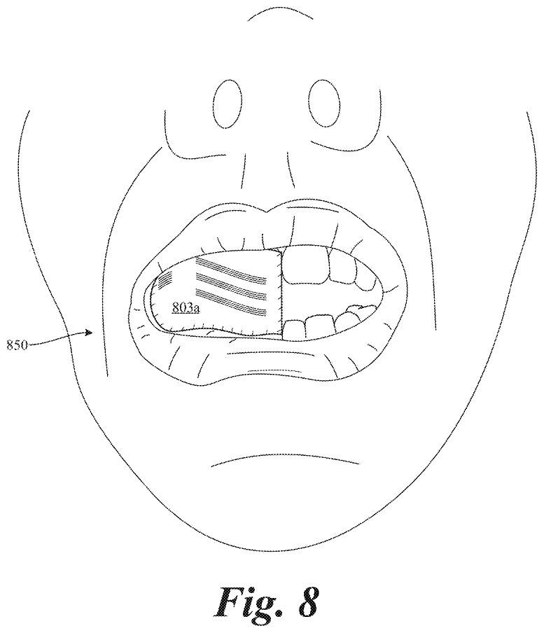

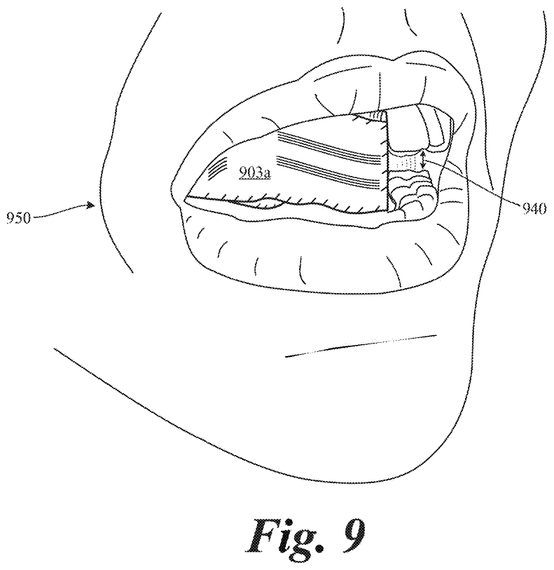

FIG. 8 depicts an embodiment of the present invention as worn by a user. Where a user is suffering from a tooth ailment, the user's pain may be pacified by restricting jaw movement, preventing contact of the upper and lower teeth. To accomplish this, the user may install the bite block on the side opposite from where the pain is occurring. For example, as depicted, the user installs the device on side 850 opposite the problematic tooth or teeth. By doing so, a gap 940 is produced, as depicted in FIG. 9. This gap is formed as a result of the thickness of the bite blocker, discussed above with respect to FIG. 5 for example. Because of the thickness of the bite block, the user is prevented from reducing the gap 940 such that opposing teeth touch. Referring back to FIG. 5, the gap 940 may be of various sizes, but is ideally from 5-7 mm depending on the user.

Additional alternative examples of the bite block are possible beyond those depicted, for example, the CTHBB may be constructed of a material which aids the user. For example, the material may be glow in the dark, or may be of a low enough density to float.

Certain more complex variations of the CTHBB may include integrated sensors in order to relay information to the patient or doctor. For example, temperature, bite pressure, moisture, tooth decay, bacteria presence, etc. For example, the CTHBB may be capable of sending small amounts of energy through a tooth in order to measure for any decay or cavities. Alternatively, a sensor may detect high levels of various bacteria, providing doctors with additional information. In these embodiments, the CTHBB may also include integrated wireless transceivers, such as a Bluetooth chipset, for sending and receiving data related to the sensed conditions to nearby computers and devices for review.

In some cases, it may be beneficial to form the bite blocker to cover more or less than half of the patient's teeth. For example, if one or more of a patient's front teeth become abscessed or are otherwise in pain, it may be beneficial to either form a bit blocker (pre-formed) that corresponds to part of one side of the patient's molars or back teeth, or two bite blockers that sit on either side of the tooth or teeth at issue. In other cases, it may be beneficial to form the bite blocker to cover more than half of the patient's teeth to further ensure that no contact is made with the tooth or teeth in pain (e.g., a % teeth bite blocker). In yet some cases, a smaller bite blocker device may be used to increase patient comfort, such as only corresponding to a third or quarter or any other fraction of the patient's teeth, for example based on location of the affected tooth. In some cases, the thickness of the anterior and/or posterior sections of the bite blocker that fit between the patient's teeth may be adjusted to account for the different amount of teeth that the bite blocker is positioned between or the intended location of the bite blocker. For example, if a smaller bite blocker is used, the thickness may be increased to better ensure that the affected tooth or teeth do not come in contact with other teeth. In additional examples, the posterior portion may include an elevated ridge in order to secure a better fit.

In additional embodiments, the Bite Block may be placed over the problematic tooth by forming a bridge over the affected tooth. This may be accomplished either as a two piece embodiment or as a single piece where there is no contact at the point over the tooth. This type of embodiment may be necessary if there are more than one affected tooth and they happen to be on opposite sides, or if there is some other reason that a bite block cannot be installed in the traditional, non-pain-side location.

In some cases, it may also be beneficial to apply or include as an additive, an ointment, medicine, antiseptic, or local anesthesia to the bite blocker. For example, pain reduction may be achieved by applying local anesthesia to the bite blocker. In such embodiments, the bite blocker may include small channels to receive and control the rate of exposure to the anesthesia. In alternative examples, the bite blocker may include additives within its construction such that the additive is released during wearing.

While various aspects of the present disclosure have been illustrated and described, as noted above, many changes can be made without departing from the spirit and scope of the disclosure. Accordingly, the scope of the disclosure is not limited by the disclosure of the above examples.

What is claimed is:

1. A method for treating a patient having an infected, diseased or damaged tooth, comprising:
selecting a predetermined volume of deformable thermoplastic material for use as a customizable oral device for inserting in the mouth of the patient;
placing the predetermined volume of deformable thermoplastic material in a predetermined volume of heated water for a first known period of time, said first known period of time sufficient to allow the deformable thermoplastic material to reach deformable plasticity;
removing the deformable thermoplastic material from the predetermined volume of heated water once the deformable thermoplastic material reaches a deformable state;
cooling the deformable thermoplastic material for a second known period of time;
installing the deformable thermoplastic material in the mouth of the patient once it has cooled beyond the second known period of time;
wherein, once the deformable thermoplastic material has been installed in the mouth of the patient for a predetermined period of time, the deformable thermoplastic material becomes plastically rigid about one or more teeth of the patient such that the deformable thermoplastic material comprises:
a channel, the channel configured to follow and engage at least a first portion of the patient's teeth;
a base portion opposite the channel, the base portion configured to follow and engage at least a second portion of the patient's teeth opposite the first portion of the patient's teeth, wherein the base portion is separated from the channel by a gap between the at least a first portion of the patient's teeth and the at least a second portion of the patient's teeth;
at least two walls extending from opposite lateral sides of the channel and in contact with at least a portion of the patient's gums; and
wherein the deformable thermoplastic material, once placed in the mouth of the patient, substantially surrounds the at least a first portion of the patient's teeth and prevents contact with the at least a first portion of the patient's teeth with the at least a second portion of the patient's teeth by a distance no less than the gap between the at least a first portion of the patient's teeth and the at least a second portion of the patient's teeth.

2. The method of claim 1 wherein the steps of claim 1 are repeated for preparing a second deformable thermoplastic material for placement on the opposite side of the patient's mouth as the at least a first portion and at least a second portion of the patient's teeth.

3. The method of claim 1, further comprising the step of applying an adhesive material to the inner channel of the deformable thermoplastic material prior to the step of installing the deformable thermoplastic material in the mouth of the patient.

4. The method of claim 3, further comprising the step of holding the deformable thermoplastic material in the desired place in the mouth of the patient to assure the deformable thermoplastic material does not move within the mouth of the patient during the cooling step.

5. The method of claim 1, wherein gap between the at least a first portion of the patient's teeth and the at least a second portion of the patient's teeth is at least 4 mm.

6. The method of claim 1, wherein the predetermined volume of heated water is heated to a temperature of no less than the boiling point of water, and the first known period of time is at least five minutes.

7. The method of claim 1, wherein the deformable thermoplastic material is comprised of at least a polypropylene.

8. The method of claim 1, wherein the cooled deformable thermoplastic material comprises a lower surface contoured to match the surfaces of the at least a second portion of teeth of the patient.

9. The method of claim 1, wherein the gap between the at least a first portion of the patient's teeth and the at least a second portion of the patient's teeth is in the range of about 4 mm to about 7 mm.

10. The method of claim 1, wherein the deformable thermoplastic material is formed by the patient.

11. The method of claim 1, wherein the deformable thermoplastic material is formed by a medical or dental professional or assistant.

12. A single piece thermoplastic material formed by the method of claim 1, comprising:
a channel, the channel configured to follow and engage at least a first portion of the patient's teeth;
a base portion opposite the channel, the base portion configured to follow and engage at least a second portion of the patient's teeth opposite the first portion of the patient's teeth, wherein the base portion is separated from the channel by a gap between the at least a first portion of the patient's teeth and the at least a second portion of the patient's teeth;
at least two walls extending from opposite lateral sides of the channel and in contact with at least a portion of the patient's gums;
wherein the deformable thermoplastic material is comprised of at least a polypropylene;
wherein the deformable thermoplastic material, following the cooling step, comprises a lower surface contoured to match the surfaces of the at least a second portion of teeth of the patient;
wherein the gap between the at least a first portion of the patient's teeth and the at least a second portion of the patient's teeth is at least 4 mm;
wherein the deformable thermoplastic material, once placed in the mouth of the patient, substantially surrounds the at least a first portion of the patient's teeth and prevents contact with the at least a first portion of the patient's teeth with the at least a second portion of the patient's teeth by a distance no less than the gap between the at least a first portion of the patient's teeth and the at least a second portion of the patient's teeth.

13. The single piece thermoplastic material of claim 12, wherein the material engages no greater than % of the first portion of the patient's teeth and the second portion of the patient's teeth.

14. The single piece thermoplastic material of claim 12, wherein the gap between the at least a first portion of the patient's teeth and the at least a second portion of the patient's teeth is in the range of 5-7 mm.

15. The deformable thermoplastic material of claim 12, wherein the material comprises at least a first non-thermoplastic material and a second thermoplastic material, the second thermoplastic material located adjacent the first portion of the patient's teeth and the second portion of the patient's teeth when installed in the patient's mouth.

16. The deformable thermoplastic material of claim 12, wherein the deformable thermoplastic material is formed by the patient.

17. The deformable thermoplastic material of claim 12, wherein the deformable thermoplastic material is formed by a dental technician or dental assistant.

18. The deformable thermoplastic material of claim 12, wherein an adhesive material is applied, immediately prior to the installation step, to the inner channel of the deformable thermoplastic material.
